(19) United States
(12) Patent Application Publication
(10) Pub. No.: US 2025/0320209 A1
(43) Pub. Date: Oct. 16, 2025

(54) Salts and Solid Forms of Sonrotoclax Intermediate

(71) Applicant: BeOne Medicines I GmbH, Basel (CH)

(72) Inventors: Liwen Xu, Suzhou (CN); Kai Peng, Suzhou (CN); Quanyou Yao, Suzhou (CN); Juntao Liu, Suzhou (CN); Qun Jiang, Suzhou (CN)

(73) Assignee: BeOne Medicines I GmbH, Basel (CH)

(21) Appl. No.: 19/250,670

(22) Filed: Jun. 26, 2025

Related U.S. Application Data
(63) Continuation of application No. PCT/CN2023/141989, filed on Dec. 26, 2023.

(30) Foreign Application Priority Data
Dec. 27, 2022 (WO) PCTCN2022142316

(51) Int. Cl. C07D 471/04

(52) U.S. Cl. CPC C07D 471/04

(57) ABSTRACT

Disclosed herein are salts and solid forms of sonrotoclax intermediate and processes for its preparation.

### CROSS-REFERENCE TO RELATED APPLICATIONS

This application is a continuation of International Application No. PCT/CN2023/141989, filed on Dec. 26, 2023, which claims priority to International Application No. PCT/CN2022/142316, filed on Dec. 27, 2022, the disclosures of each of which are hereby incorporated by reference in their entireties.

### FIELD

Disclosed herein are salts and solid forms of sonrotoclax intermediate and processes for making sonrotoclax.

### BACKGROUND

International publication WO2019/210828 disclosed a series of Bcl-2 inhibitors, in particular, 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (hereinafter sonrotoclax).

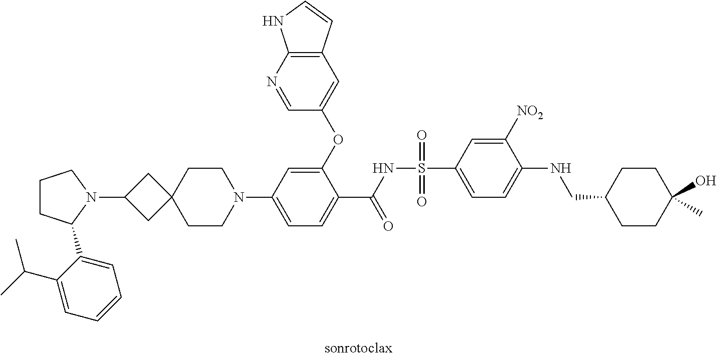 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

The process of making sonrotoclax disclosed therein is not ideal for scale-up as the intermediate methyl 4-(2-{(2S)-2-[2-(propan-2-yl)phenyl]pyrrolidin-1-yl}-7-azaspiro[3.5]nonan-7-yl)-2-[(1H-pyrrolo[2,3-b]pyridin-5-yl)oxy]benzoate (Compound 2) was obtained as an oil or liquid with low chemical purity. Thus, a new process for making sonrotoclax is desirable.

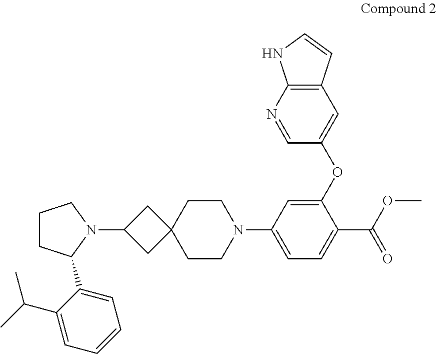 [SMILES: COC(=O)c1ccc(N2CCC3(CC2)CC(N2CCC[C@H]2c2ccccc2C(C)C)C3)cc1Oc1cnc2[nH]ccc2c1]

### SUMMARY

Provided herein are salts of Compound 2, and crystalline forms, and amorphous forms thereof. Further provided herein are methods of making the salts of Compound 2, and crystalline forms and amorphous forms thereof. Further provided herein are methods of making sonrotoclax using the salts of Compound 2, and crystalline forms and amorphous forms thereof.

### BRIEF DESCRIPTION OF THE FIGURES

FIG. 1A shows the XRPD pattern of oxalate Type A.

FIG. 1B shows TGA/DSC curves of oxalate Type A.

FIG. 1C shows NMR spectrum of oxalate Type A.

FIG. 2A shows the XRPD pattern of oxalate Type B.

FIG. 2B shows TGA/DSC curves of oxalate Type B.

FIG. 2C shows NMR spectrum of oxalate Type B.

FIG. 3A shows the XRPD pattern of oxalate Type C.

FIG. 3B shows TGA/DSC curves of oxalate Type C.

FIG. 3C shows NMR spectrum of oxalate Type C.

FIG. 4A shows the XRPD pattern of oxalate Type D.

FIG. 4B shows TGA/DSC curves of oxalate Type D.

FIG. 4C shows NMR spectrum of oxalate Type D.

### DETAILED DESCRIPTION

Unless defined otherwise, all technical and scientific terms used herein have the same meaning as is commonly understood by one of skill in the art. All patents, patent applications, and publications referred to herein are incorporated by reference.

As used herein, the term “solvate” refers to a crystalline form of Compound 2 which contains solvent.

As used herein, the term “crystal form” or “crystalline form” refers to a solid form that is crystalline. In certain embodiments, a crystal form of a substance may be substantially free of amorphous forms and/or other crystal forms. In certain embodiments, a crystal form of a substance may contain less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 35%, less than about 40%, less than about 45%, or less than about 50% by weight of one or more amorphous forms and/or other crystal forms. In certain embodiments, a crystal form of a substance may be physically and/or chemically pure. In certain embodiments, a crystal form of a substance may be about 99%, about 98%, about 97%, about 96%, about 95%, about 94%, about 93%, about 92%, about 91%, or about 90% physically and/or chemically pure.

As used herein, and unless otherwise specified, the term “amorphous” or “amorphous form” means that the substance, component, or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In certain embodiments, an amorphous form of a substance may contain less than about 1%, less than about 2%, less than about 3%, less than about 4%, less than about 5%, less than about 10%, less than about 15%, less than about 20%, less than about 25%, less than about 30%, less than about 35%, less than about 40%, less than about 45%, or less than about 50% by weight of one or more other amorphous forms and/or crystal forms on a weight basis. In certain embodiments, an amorphous form of a substance may be physically and/or chemically pure. In certain embodiments, an amorphous form of a substance be about 99%, about 98%, about 97%, about 96%, about 95%, about 94%, about 93%, about 92%, about 91%, or about 90% physically and/or chemically pure.

As used herein, the term “about” when used in reference to XRPD peak positions refers to the inherent variability of peaks depending on the calibration of the instrument, processes used to prepare the crystalline forms of the present disclosure, age of the crystalline forms and the type of instrument used in the analysis. The variability of the instrumentation used for XRPD analysis was about ±0.2° 20.

As used herein, the term “about” when used in reference to DSC endothermic peak onset refers to the inherent variability of peaks depending on the calibration of the instrument, method used to prepare the samples of the present disclosure, and the type of instrument used in the analysis. The variability of the instrumentation used for DSC analysis was about ±1° C.

The term “about” as used herein other than the former definition, unless indicated otherwise, denotes that a number (e.g., temperature, pH, volume, etc.) can vary within ±10%, preferably within ±5%.

Experimental measurements, results, or observations, such as XRPD patterns, DSC thermograms, NMR spectra, DVS isotherms or TGA thermal curves, are said to be “substantially in accordance” with another XRPD pattern, DSC thermogram, NMR spectra, DVS isotherm or TGA thermal curve when one skilled in the art would consider them to represent the same single crystalline form of the same compound. Thus, an XRPD pattern, DSC thermogram, NMR spectra, DVS isotherm or TGA thermal curve that is substantially in accordance with one or more figures provided herein may be identical or, more likely, may be somewhat different. For example, an XRPD pattern that is somewhat different from one or more of the figures may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern, DSC thermogram, NMR spectra, DVS isotherm, or TGA thermal curve of the sample and the corresponding XRPD pattern, DSC thermogram, NMR spectra, DVS isotherm or TGA thermal curve disclosed herein.

Surprisingly, salts of Compound 2, preferably oxalate salt of Compound 2, even more preferably the crystalline of oxalate salt of Compound 2 is a solid with very low viscosity and very good stability. The crystalline form of the oxalate salt of Compound 2, especially Form B of the oxalate can be obtained with a very good purity without further purification, which is suitable for large-scale industrial process. The particle size of Form B is 1-50 μm, preferably is 1-30 μm, more preferably is 5-20 μm, even more preferably is 5-10 μm. Such particle size can be obtained by filtration easily and quickly.

Aspect 1. A salt of formula (I):

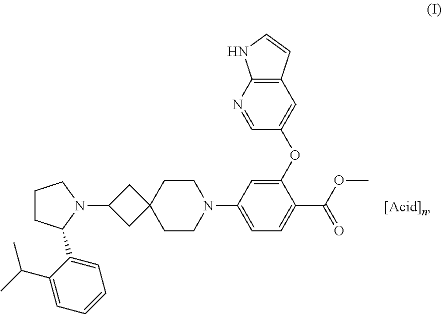 [SMILES: COC(=O)c1ccc(N2CCC3(CC2)CC(N2CCC[C@H]2c2ccccc2C(C)C)C3)cc1Oc1cnc2[nH]ccc2c1]

wherein [Acid] is selected from hydrochloric acid, sulfuric acid, phosphoric acid, hydrobromic acid, nitric acid, fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
n is about 0.5 to about 3,
preferably, n is 0.5-2; more preferably, n is 0.5-1.5; even more preferably, n is 1. In certain embodiments, n is 0.5, 1.0±0.1, 1.5±0.1, 2.0±0.1, 2.5±0.1, or 3.0.

Aspect 2. The salt of Aspect 1, wherein [Acid] is selected from hydrochloric acid, sulfuric acid, phosphoric acid, hydrobromic acid and/or nitric acid.

Aspect 3. The salt of Aspect 1, wherein [Acid] is selected from tartaric acid (L-tartaric acid or D-tartaric acid), malic acid, citric acid, p-toluenesulfonic acid, D-DTTA, Maleic acid and/or oxalic acid; preferably, [Acid] is selected from oxalic acid.

Aspect 4. The salt of Aspect 1, which the salt is an oxalate salt of Formula (II):

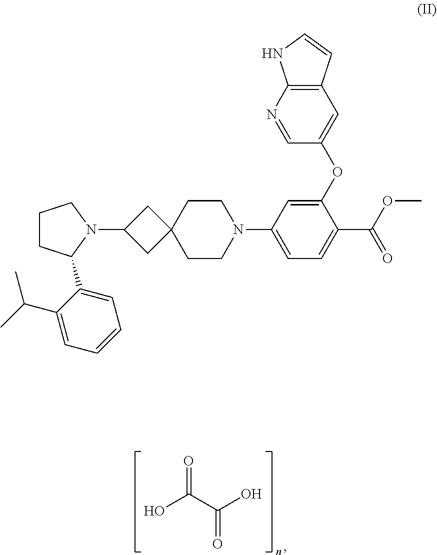

wherein n is about 0.5 to about 3;
preferably, n is 0.5-2; more preferably, n is 0.5-1.5; even more preferably, n is 0.5, 1.0±0.1, 1.5±0.1, 2.0±0.1, 2.5±0.1, 3.0; even more preferably, n is 1.0
even more preferably, the salt is an oxalate salt of Formula (III):

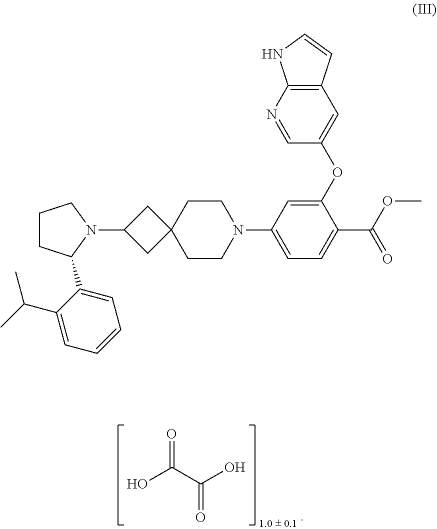

Aspect 5. A crystalline form of a compound of Formula IV

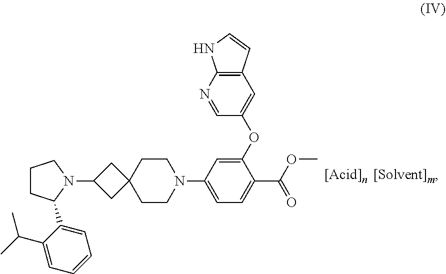 [SMILES: COC(=O)c1ccc(N2CCC3(CC2)CC(N2CCC[C@H]2c2ccccc2C(C)C)C3)cc1Oc1cnc2[nH]ccc2c1]

wherein [Acid] is selected from the group consisting of hydrochloric acid, sulfuric acid, phosphoric acid, hydrobromic acid, nitric acid, fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
[Solvent] is selected from H₂O or organic solvents;
n is a number from about 0.5 to about 3;
m is a number from about 0.0 to about 5.0.

Aspect 6. The crystalline form of Aspect 5, wherein [Acid] is selected from the group consisting of inorganic acid selected from Hydrochloric acid, sulfuric acid, phosphoric acid, hydrobromic acid and/or nitric acid; or organic acid selected from fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
preferably [Acid] is selected from tartaric acid (L-tartaric acid or D-tartaric acid), malic acid, citric acid, p-toluenesulfonic acid, D-DTTA, Maleic acid and/or oxalic acid;
more preferably [Acid] is selected from oxalic acid.

Aspect 7. The crystalline form of any one of Aspects 5-6, wherein n is about 0.5 to about 3;
preferably, n is 0.5-2; more preferably, n is 0.5-1.5; even more preferably, n is 0.5, 1.0±0.1, 1.5±0.1, 2.0±0.1, 2.5±0.1, 3.0; even more preferably, n is 1.0.

Aspect 8. The crystalline form of any one of Aspects 5-7, wherein the [solvent] is selected from MeOH, EtOH, i-PrOH, n-PrOH, n-BuOH, t-BuOH, DCM, CPME, toluene, acetone, butanone, pentanone, H₂O, MeCN, THF, ether, propyl ether, n-heptane, hexane, 1,4-dioxane or EtOAc.

Aspect 9. The crystalline form of any one of Aspects 5-8, wherein m is a number about 0.0 to about 3.0, preferably about 0.0 to about 2.0, more preferably m is a number selected from the group consisting of 0.1±0.1, 0.5±0.1, 1.0±0.2, 1.5±0.2 and 2.0±0.2, even more preferably, m is 0˜0.2, 0.95˜1.05, 1.05˜1.15, 1.45˜1.55, 1.90˜2.10; more preferably, m is 0.98˜1.02, 1.08˜1.12 or 1.48˜1.52, 1.95˜2.15; even more preferably, m is 0, 0.1, 0.2, 1.0, 1.5, 2.0, 2.5, 3.0, 3.5, 4.0, 4.5, 5.0, 5.5, 6.0, 6.5 or 7.0.

Aspect 10. A crystalline form of the oxalate salt of Compound 2, which is
(a) Crystalline Form A, characterized by a powder X-ray diffraction pattern comprising three, four, five, six, seven, eight, nine or more diffraction peaks having 2θ angle values independently selected from the group consisting of 3.89±0.2, 7.69±0.2, 10.55±0.2, 10.84±0.2, 11.21±0.2, 11.68±0.2, 12.31±0.2, 13.02±0.2, 13.83±0.2, 14.69±0.2, 15.36±0.2, 15.58±0.2, 16.60±0.2, 17.58±0.2, 18.39±0.2, 18.98±0.2, 19.32±0.2, 19.81±0.2, 20.37±0.2, 21.05±0.2, 21.61±0.2, 21.91±0.2, 22.49±0.2, 23.09±0.2, 23.48±0.2, 24.15±0.2, 24.76±0.2, 25.44±0.2, 25.66±0.2, 26.18±0.2, 26.67±0.2, 27.00±0.2, 28.11±0.2, 28.71±0.2, 29.09±0.2, 29.55±0.2, 29.91±0.2, 31.52±0.2, 32.18±0.2, 32.80±0.2, 34.49±0.2, 34.97±0.2, 36.66±0.2, 38.52±0.2, 39.32±0.2, and 39.71±0.2; or
(b) Crystalline Form B, characterized by a powder X-ray diffraction pattern comprising three, four, five, six, seven, eight, nine or more diffraction peaks having 2θ angle values independently selected from the group consisting of 4.05±0.2, 8.00±0.2, 10.79±0.2, 11.23±0.2, 11.80±0.2, 12.02±0.2, 12.45±0.2, 12.95±0.2, 13.92±0.2, 15.55±0.2, 16.03±0.2, 16.83±0.2, 17.14±0.2, 17.76±0.2, 18.41±0.2, 19.38±0.2, 20.08±0.2, 20.50±0.2, 21.04±0.2, 21.66±0.2, 22.08±0.2, 22.52±0.2, 23.34±0.2, 23.72±0.2, 24.09±0.2, 24.82±0.2, 25.31±0.2, 25.66±0.2, 26.02±0.2, 27.21±0.2, 27.78±0.2, 28.21±0.2, 28.53±0.2, 29.01±0.2, 31.06±0.2, 31.45±0.2, 32.28±0.2, 33.02±0.2, 34.67±0.2, 35.30±0.2, 36.02±0.2, and 38.00±0.2; or
(c) Crystalline Form C, characterized by a powder X-ray diffraction pattern comprising three, four, five, six, seven, eight, nine or more diffraction peaks having 2θ angle values independently selected from the group consisting of 3.80±0.2, 4.01±0.2, 8.00±0.2, 10.42±0.2, 11.31±0.2, 11.55±0.2, 12.02±0.2, 12.94±0.2, 13.91±0.2, 14.13±0.2, 14.61±0.2, 16.04±0.2, 16.93±0.2, 17.76±0.2, 18.43±0.2, 18.91±0.2, 19.91±0.2, 20.29±0.2, 20.96±0.2, 21.53±0.2, 21.87±0.2, 22.34±0.2, 22.62±0.2, 23.16±0.2, 23.55±0.2, 23.76±0.2, 24.18±0.2, 24.86±0.2, 25.39±0.2, 25.65±0.2, 26.04±0.2, 26.46±0.2, 26.96±0.2, 27.53±0.2, 28.03±0.2, 28.47±0.2, 29.02±0.2, 29.47±0.2, 29.70±0.2, 30.16±0.2, 30.98±0.2, 31.36±0.2, 31.87±0.2, 32.53±0.2, 33.38±0.2, 34.41±0.2, 35.63±0.2, 35.91±0.2, 36.59±0.2, 38.15±0.2, and 38.66±0.2; or
(d) Crystalline Form D, characterized by a powder X-ray diffraction pattern comprising three, four, five, six, seven, eight, nine or more diffraction peaks having 2θ angle values independently selected from the group consisting of 4.06±0.2, 8.03±0.2, 10.51±0.2, 10.84±0.2, 11.60±0.2, 12.06±0.2, 12.85±0.2, 14.04±0.2, 14.67±0.2, 16.08±0.2, 16.98±0.2, 17.36±0.2, 17.80±0.2, 18.36±0.2, 18.68±0.2, 19.15±0.2, 19.85±0.2, 20.20±0.2, 20.46±0.2, 21.20±0.2, 21.66±0.2, 21.97±0.2, 22.38±0.2, 23.31±0.2, 23.80±0.2, 24.14±0.2, 24.31±0.2, 24.93±0.2, 25.52±0.2, 26.77±0.2, 27.28±0.2, 27.48±0.2, 28.33±0.2, 28.75±0.2, 29.59±0.2, 30.10±0.2, 30.81±0.2, 31.20±0.2, 32.16±0.2, 32.61±0.2, 33.36±0.2, 36.05±0.2, 38.64±0.2, and 39.25±0.2.

Aspect 11. A crystalline form of the oxalate salt of Compound 2, which is
(a) Crystalline Form A, characterized by a powder X-ray diffraction pattern comprising diffraction peaks having 2θ angle values of 11.68±0.2, 15.36±0.2, and 21.61±0.2;
preferably having 2θ angle values of 10.55±0.2, 11.68±0.2, 12.31±0.2, 15.36±0.2, 21.05±0.2, and 21.61±0.2;
more preferably having 2θ angle values of 10.55±0.2, 11.68±0.2, 12.31±0.2, 15.36±0.2, 15.58±0.2, 21.05±0.2, 21.61±0.2, 21.91±0.2, and 23.48±0.2;
even more preferably having 2θ angle values of 10.55±0.2, 11.68±0.2, 12.31±0.2, 15.36±0.2, 15.58±0.2, 18.39±0.2, 19.81±0.2, 21.05±0.2, 21.61±0.2, 21.91±0.2, 23.48±0.2, and 25.44±0.2;
even more preferably having 2θ angle values of 7.69±0.2, 10.55±0.2, 11.68±0.2, 11.21±0.2, 12.31±0.2, 15.36±0.2, 15.58±0.2, 18.39±0.2, 19.81±0.2, 21.05±0.2, 21.61±0.2, 21.91±0.2, 22.49±0.2, 23.48±0.2, and 25.44±0.2; or
(b) Crystalline Form B, characterized by a powder X-ray diffraction pattern comprising diffraction peaks having 2θ angle values of 11.80±0.2, 12.02±0.2, and 16.03±0.2;
preferably having 2θ angle values of 8.00±0.2, 11.80±0.2, 12.02±0.2, 12.45±0.2, 16.03±0.2, and 20.50±0.2;
more preferably having 2θ angle values of 8.00±0.2, 10.79±0.2, 11.80±0.2, 12.02±0.2, 12.45±0.2, 16.03±0.2, 20.50±0.2, 23.72±0.2, and 26.02±0.2;
even more preferably having 2θ angle values of 8.00±0.2, 10.79±0.2, 11.80±0.2, 12.02±0.2, 12.45±0.2, 16.03±0.2, 16.83±0.2, 20.08±0.2, 20.50±0.2, 23.72±0.2, 24.09±0.2, and 26.02±0.2;
even more preferably having 2θ angle values of 8.00±0.2, 10.79±0.2, 11.80±0.2, 12.02±0.2, 12.45±0.2, 13.92±0.2, 15.55±0.2, 16.03±0.2, 16.83±0.2, 20.08±0.2, 20.50±0.2, 23.72±0.2, 24.09±0.2, 26.02±0.2, and 28.21±0.2; or
(c) Crystalline Form C, characterized by a powder X-ray diffraction pattern comprising diffraction peaks having 2θ angle values of 10.42±0.2, 12.94±0.2, and 20.29±0.2; preferably having 2θ angle values of 10.42±0.2, 12.94±0.2, 16.04±0.2, 18.91±0.2, 19.91±0.2, and 20.29±0.2;
more preferably having 2θ angle values of 10.42±0.2, 12.02±0.2, 12.94±0.2, 16.04±0.2, 16.93±0.2, 18.91±0.2, 19.91±0.2, 20.29±0.2, and 24.18±0.2;
even more preferably having 2θ angle values of 10.42±0.2, 12.02±0.2, 12.94±0.2, 16.04±0.2, 16.93±0.2, 18.91±0.2, 19.91±0.2, 20.29±0.2, 20.96±0.2, 21.87±0.2, 23.16±0.2, and 24.18±0.2;
even more preferably having 2θ angle values of 10.42±0.2, 12.02±0.2, 12.94±0.2, 16.04±0.2, 16.93±0.2, 18.43±0.2, 18.91±0.2, 19.91±0.2, 20.29±0.2, 20.96±0.2, 21.53±0.2, 21.87±0.2, 23.16±0.2, 24.18±0.2, and 28.47±0.2; or
(d) Crystalline Form D, characterized by a powder X-ray diffraction pattern comprising diffraction peaks having 2θ angle values of 10.51±0.2, 20.20±0.2, and 20.46±0.2;
preferably having 2θ angle values of 10.51±0.2, 12.06±0.2, 12.85±0.2, and 16.08±0.2, 20.20±0.2, 20.46±0.2;
more preferably having 2θ angle values of 10.51±0.2, 12.06±0.2, 12.85±0.2, 16.08±0.2, 16.98±0.2, 20.20±0.2, 20.46±0.2, 21.66±0.2, and 23.80±0.2;
even more preferably having 2θ angle values of 10.51±0.2, 10.84±0.2, 12.06±0.2, 12.85±0.2, 16.08±0.2, 16.98±0.2, 19.15±0.2, 19.85±0.2, 20.20±0.2, 20.46±0.2, 21.66±0.2, and 23.80±0.2;
even more preferably having 2θ angle values of 10.51±0.2, 10.84±0.2, 12.06±0.2, 12.85±0.2, 16.08±0.2, 16.98±0.2, 18.36±0.2, 19.15±0.2, 19.85±0.2, 20.20±0.2, 20.46±0.2, 21.20±0.2, 21.66±0.2, 22.38±0.2, and 23.80±0.2.

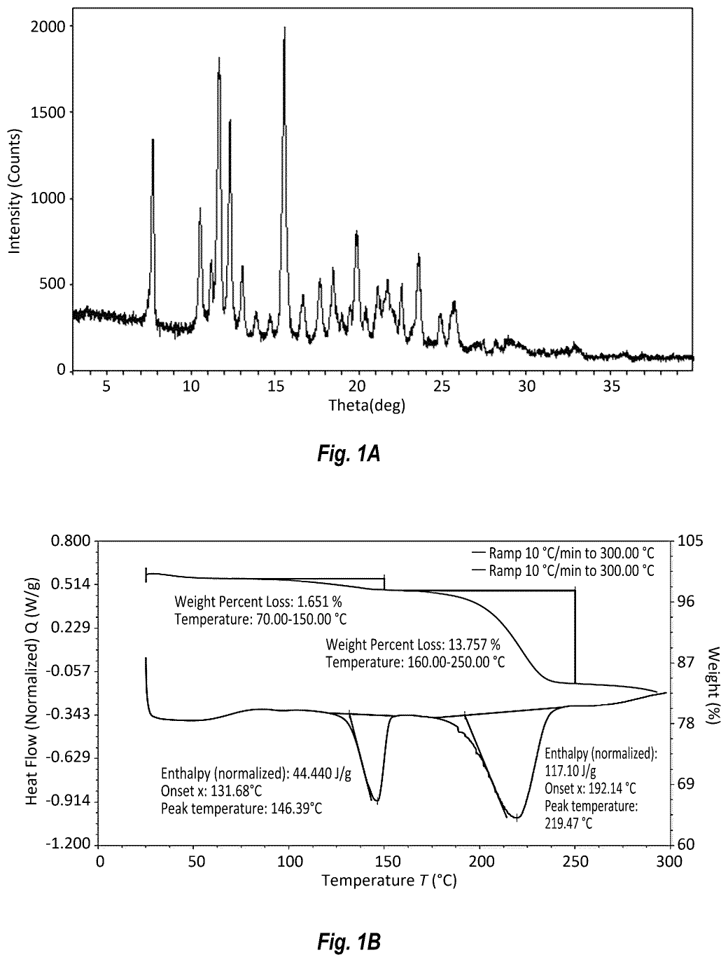

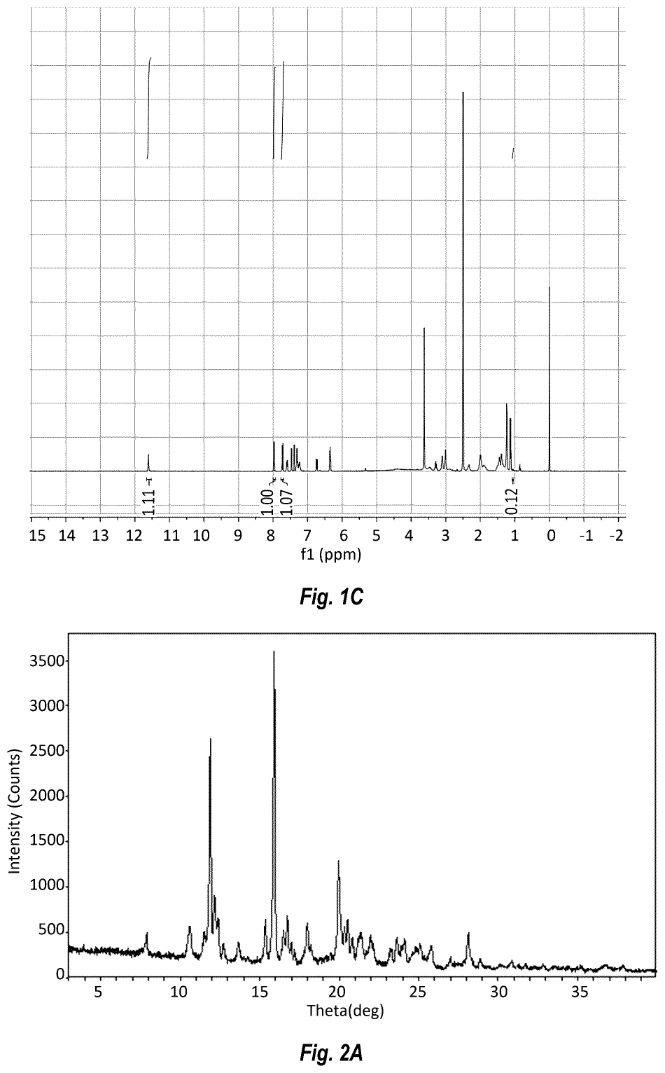

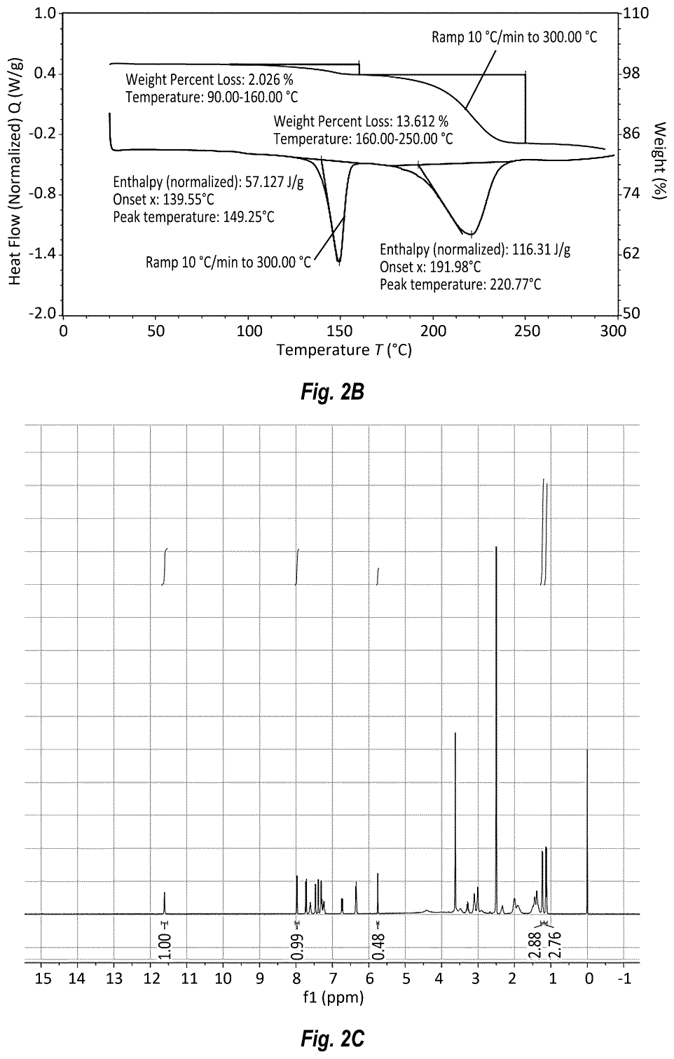

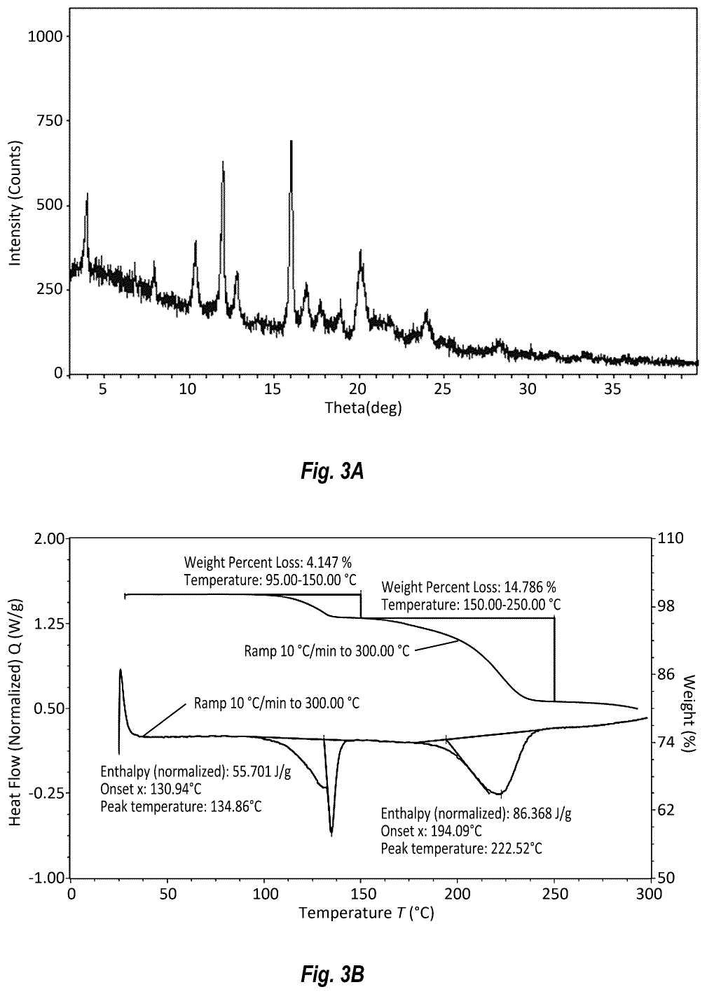

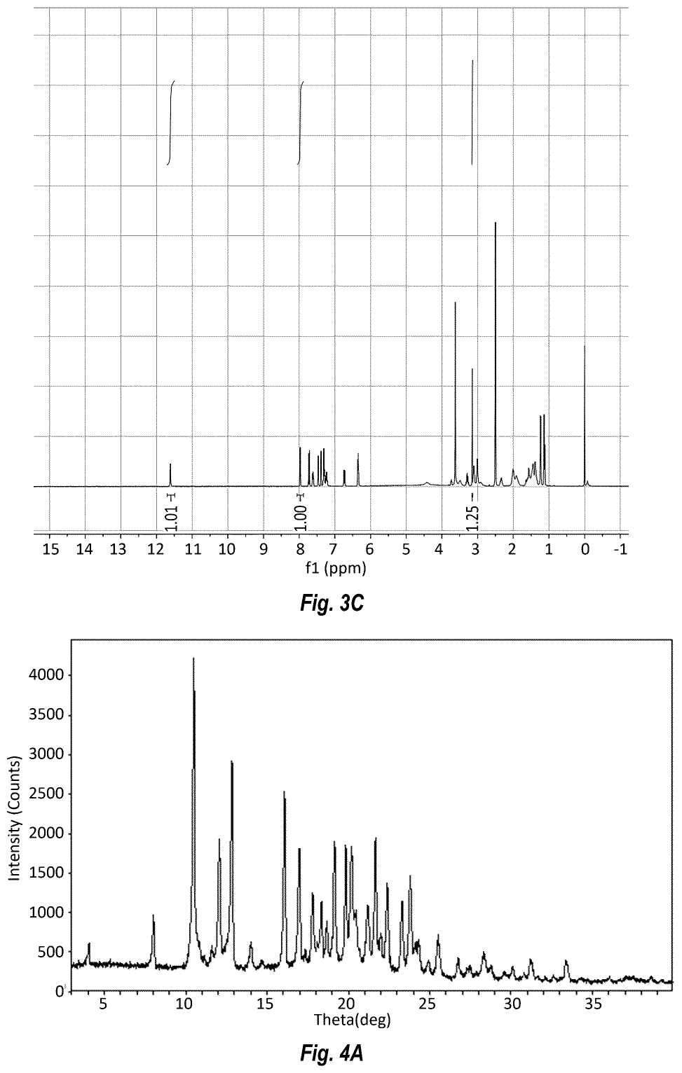

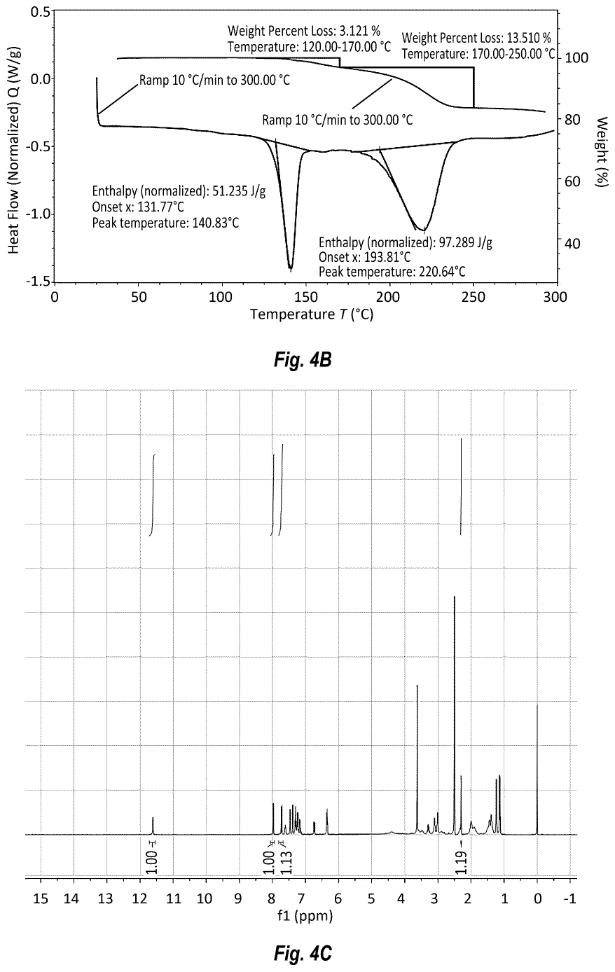

Aspect 12. A crystalline form of the oxalate salt of Compound 2, characterized by a powder X-ray diffraction pattern substantially in accordance with that depicted in a figure selected from the group consisting of FIG. 1A, FIG. 2A, FIG. 3A, and FIG. 4A.

Aspect 13. A process for preparing a crystalline form of the oxalate salt of Compound 2, comprising:
Procedure (1) dissolving the oxalate salt according to Aspect 4 in EtOH, and evaporating slowly the resultant solution to give the crystalline form; or
Procedure (2) dissolving the oxalate salt according to Aspect 4 in DCM, and evaporating slowly the resultant solution to give the crystalline form; or
Procedure (3) suspending the oxalate salt according to Aspect 4 in CPME, and stirring the resultant mixture to give the crystalline form; or
Procedure (4) suspending the oxalate salt according to Aspect 4 in toluene, and stirring the mixture to give the crystalline form,
preferably wherein the oxalate salt according to Aspect 4 is in amorphous form.

Aspect 14. The process for preparing a crystalline form of Aspect 13, wherein the time of Procedure (1) is 0-10 days, preferably is 0-7 days, more preferably is 2-4 days, even more preferably is 1, 2, 3, 4 or 5 days;
the time of Procedure (2) 0-10 days, preferably is 0-7 days, more preferably is 2-4 days, even more preferably is 1, 2, 3, 4 or 5 days;
the time of Procedure (3) 0-10 days, preferably is 0-7 days, more preferably is 2-4 days, even more preferably is 1, 2, 3, 4, 5, 6 or 7 days;
the time of Procedure (4) 0-10 days, preferably is 0-7 days, more preferably is 2-4 days, even more preferably is 1, 2, 3, 4, 5, 6 or 7 days.

Aspect 15. The process for preparing a crystalline form of Aspect 13-14, wherein the oxalate salt/EtOH (mg/ml) in Procedure (1) is 1:1 to 100:1, preferably is 1:1 to 50:1, more preferably is 20:1 to 40:1, even more preferably is 30:1;
The oxalate salt/DCM (mg/ml) in Procedure (2) is 1:1 to 100:1, preferably is 1:1 to 50:1, more preferably is 20:1 to 40:1, even more preferably is 30:1;
The oxalate salt/CPME (mg/ml) in Procedure (3) is 1:1 to 100:1, preferably is 20:1 to 80:1, more preferably is 40:1 to 70:1, even more preferably is 60:1;
The oxalate salt/toluene (mg/ml) in Procedure (4) is 1:1 to 100:1, preferably is 20:1 to 80:1, more preferably is 40:1 to 70:1, even more preferably is 60:1.

Aspect 16. The process for preparing a crystalline form of the oxalate salt of Compound 2, wherein the temperature of Procedure (1) is 0 to 50° C., preferably is 10 to 40° C., more preferably is 20 to 30° C., even more preferably is 20 to 25° C. or room temperature;
the temperature of Procedure (2) is 0 to 50° C., preferably is 10 to 40° C., more preferably is 20 to 30° C., even more preferably is 20 to 25° C. or room temperature;
the temperature of Procedure (3) is 0 to 50° C., preferably is 10 to 40° C., more preferably is 20 to 30° C., even more preferably is 20 to 25° C. or room temperature;
the temperature of Procedure (4) is 10 to 110° C., preferably is 30 to 100° C., more preferably is 60 to 90° C., even more preferably is 80° C. or room temperature.

Aspect 17. The process for the preparation of the crystalline form of Aspect 13-16, wherein the starting material is selected from amorphous, type A, B, C, D; preferably is amorphous.

Aspect 18. The process for preparing a crystalline form of Aspect 13-17, wherein the process further comprises:
Procedure (5): dissolve the free base of compound 3 in a solvent, then add oxalic acid to obtain the oxalate salt, wherein compound 3 is

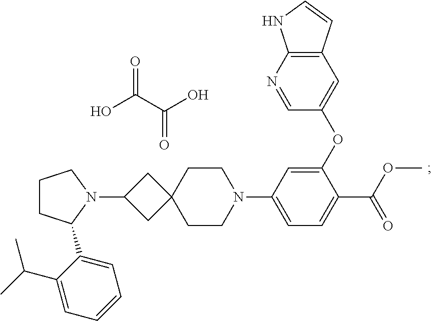

preferably, the solvent of Procedure (5) is methyl tert-butyl ether;
more preferably, oxalic acid is added by using the solution of methyl tert-butyl ether;
even more preferably, Procedure (5) is performed at room temperature or at 20° C.-30° C.

Aspect 19. The crystalline form of any one of Aspects 5-12 or the salt of any one of Aspects 1-4 is used as an intermediate of preparing sonrotoclax.

Aspect 20. Provided here is a process of preparing sonrotoclax,

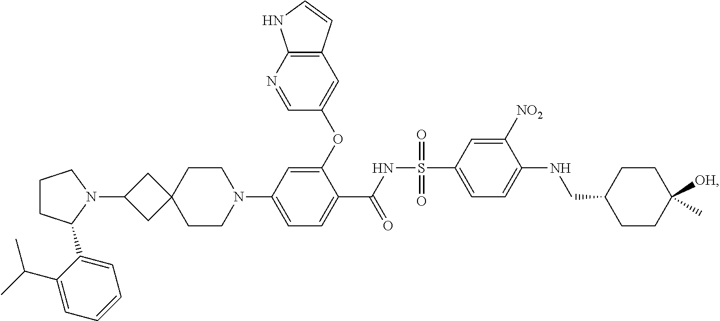 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof, comprising reacting a salt of formula (I), with 4-((((1r,4r)-4-hydroxy-4-methylcyclohexyl)methyl)amino)-3-nitrobenzenesulfonamide.

In one embodiment of Aspect 20, the reaction happens at the presence of 1-ethyl-3-(3-dimethylaminopropyl)carbodiimide (EDCI) and 4-dimethylaminopyridine (DMAP).

Aspect 21. Provided here is a method of preparing sonrotoclax,

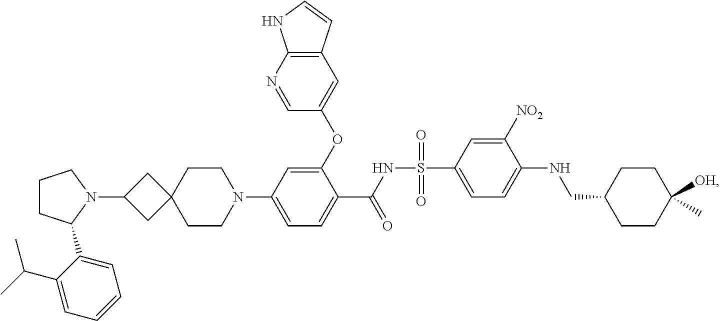 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]

or a salt thereof, comprising reacting a salt of formula (I), with an acid or a base, to provide (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid,

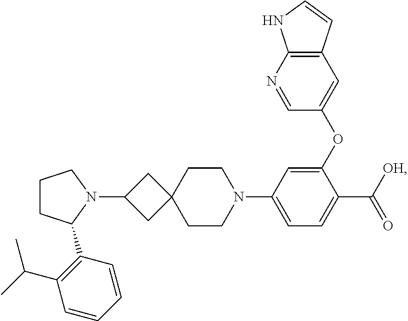 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof.

In one embodiment of Aspect 21, the acid is HCl, or the base is NaOH.

Aspect 22. Provided here is a method of making sonrotoclax comprising reacting (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid,

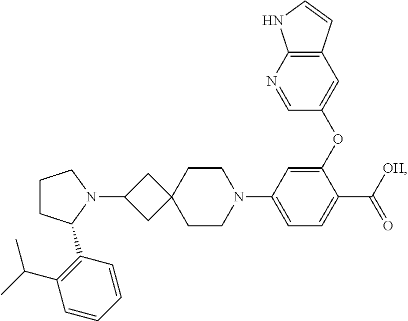 [SMILES: CC(C)c1ccccc1[C@@H]1CCCN1C1CC2(CCN(c3ccc(C(=O)O)c(Oc4cnc5[nH]ccc5c4)c3)CC2)C1]

or a salt thereof, with 4-((((1r,4r)-4-hydroxy-4-methylcyclohexyl)methyl)amino)-3-nitrobenzenesulfonamide.

In one embodiment of Aspect 22, the reaction happens at the presence of EDCI and DMAP.

In one embodiment of Aspect 23, provided here is a method for preparing a pharmaceutical composition comprising Sonrotoclax, comprising mixing Sonrotoclax with a pharmaceutically acceptable excipient, wherein Sonrotoclax is prepared according to the method provided here.

### Numbered Embodiments

Embodiment 1. A salt of formula (I):

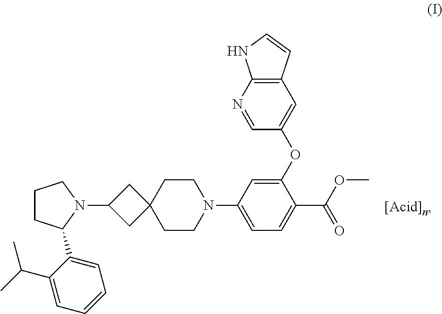 [SMILES: COC(=O)c1ccc(N2CCC3(CC2)CC(N2CCC[C@H]2c2ccccc2C(C)C)C3)cc1Oc1cnc2[nH]ccc2c1]

wherein [Acid] is selected from hydrochloric acid, sulfuric acid, phosphoric acid, hydrobromic acid, nitric acid, fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
n is about 0.5 to about 3.

Embodiment 2. The salt of Embodiment 1, wherein [Acid] is selected from hydrochloric acid, sulfuric acid, phosphoric acid, hydrobromic acid and/or nitric acid.

Embodiment 3. The salt of Embodiment 1, wherein [Acid] is selected from tartaric acid (L-tartaric acid or D-tartaric acid), malic acid, citric acid, p-toluenesulfonic acid, D-DTTA, Maleic acid and/or oxalic acid; preferably, [Acid] is selected from oxalic acid.

Embodiment 4. The salt of Embodiment 1, which the salt is an oxalate salt of Formula (II):

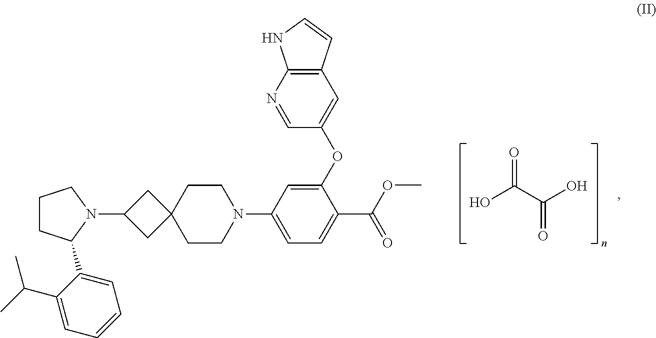

wherein n is about 0.5 to about 3;
preferably, n is 0.5-2; more preferably, n is 0.5-1.5; even more preferably, n is 0.5, 1.00±0.1, 1.5±0.1, 2.0±0.1, 2.5±0.1, 3.0; even more preferably, n is 1.0
even more preferably, the salt is an oxalate salt of Formula (III):

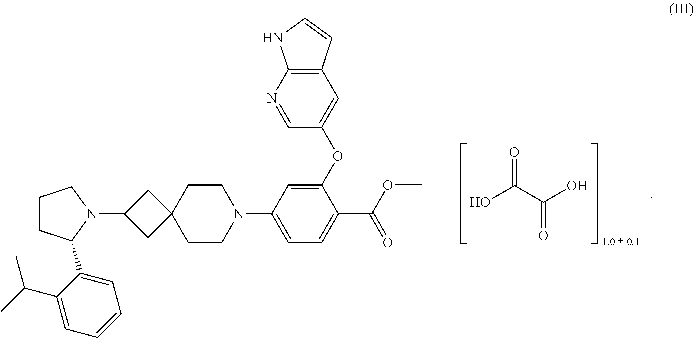

Embodiment 5. A crystalline form of a salt of Formula IV

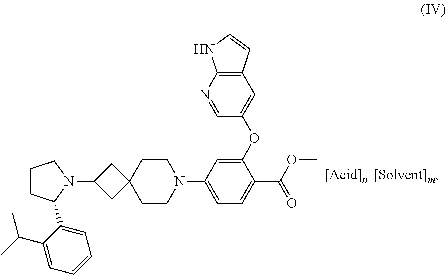 [SMILES: COC(=O)c1ccc(N2CCC3(CC2)CC(N2CCC[C@H]2c2ccccc2C(C)C)C3)cc1Oc1cnc2[nH]ccc2c1]

wherein [Acid] is selected from the group consisting of hydrochloric acid, sulfuric acid, phosphoric acid, hydrobromic acid, nitric acid, fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
[Solvent] is selected from H₂O or organic solvents;
n is a number from about 0.5 to about 3;
m is a number from about 0.0 to about 5.0.

Embodiment 6. The crystalline form of Embodiment 5, wherein [Acid] is selected from the group consisting of inorganic acid selected from Hydrochloric acid, sulfuric acid, phosphoric acid, hydrobromic acid and/or nitric acid; or organic acid selected from fumaric acid, tartaric acid (L-tartaric acid or D-tartaric acid), lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, D-DTTA, Maleic acid and/or oxalic acid;
preferably [Acid] is selected from tartaric acid (L-tartaric acid or D-tartaric acid), malic acid, citric acid, p-toluenesulfonic acid, D-DTTA, Maleic acid and/or oxalic acid;
more preferably [Acid] is selected from oxalic acid.

Embodiment 7. The crystalline form of any one of Embodiments 5-6, wherein n is about 0.5 to about 3;
preferably, n is 0.5-2; more preferably, n is 0.5-1.5; even more preferably, n is 0.5, 1.00±0.1, 1.5±0.1, 2.0±0.1, 2.5±0.1, 3.0; even more preferably, n is 1.0.

Embodiment 8. The crystalline form of any one of Embodiments 5-7, wherein the [solvent] is selected from MeOH, EtOH, i-PrOH, n-PrOH, n-BuOH, t-BuOH, DCM, CPME, toluene, acetone, butanone, pentanone, H₂O, MeCN, THF, ether, propyl ether, n-heptane, hexane, 1,4-dioxane or EtOAc.

Embodiment 9. The crystalline form of any one of Embodiments 5-8, wherein m is a number about 0.0 to about 3.0, preferably about 0.0 to about 2.0, more preferably m is a number selected from the group consisting of 0.1±0.1, 0.5±0.1, 1.0±0.2, 1.5±0.2 and 2.0±0.2, even more preferably, m is 0˜0.2, 0.95˜1.05, 1.05˜1.15, 1.45˜1.55, 1.90˜2.10; more preferably, m is 0.98˜1.02, 1.08˜1.12 or 1.48˜1.52, 1.95˜2.15; even more preferably, m is 0, 0.1, 0.2, 1.0, 1.5, 2.0, 2.5, 3.0, 3.5, 4.0, 4.5, 5.0, 5.5, 6.0, 6.5 or 7.0.

Embodiment 10. The crystalline form of any one of Embodiments 5-9, which is
(a) oxalate Crystalline Form A, characterized by a powder X-ray diffraction pattern comprising three, four, five, six, seven, eight, nine or more diffraction peaks having 2θ angle values independently selected from the group consisting of 3.89±0.2, 7.69±0.2, 10.55±0.2, 10.84±0.2, 11.21±0.2, 11.68±0.2, 12.31±0.2, 13.02±0.2, 13.83±0.2, 14.69±0.2, 15.36±0.2, 15.58±0.2, 16.60±0.2, 17.58±0.2, 18.39±0.2, 18.98±0.2, 19.32±0.2, 19.81±0.2, 20.37±0.2, 21.05±0.2, 21.61±0.2, 21.91±0.2, 22.49±0.2, 23.09±0.2, 23.48±0.2, 24.15±0.2, 24.76±0.2, 25.44±0.2, 25.66±0.2, 26.18±0.2, 26.67±0.2, 27.00±0.2, 28.11±0.2, 28.71±0.2, 29.09±0.2, 29.55±0.2, 29.91±0.2, 31.52±0.2, 32.18±0.2, 32.80±0.2, 34.49±0.2, 34.97±0.2, 36.66±0.2, 38.52±0.2, 39.32±0.2, and 39.71±0.2; or
(b) oxalate Crystalline Form B, characterized by a powder X-ray diffraction pattern comprising three, four, five, six, seven, eight, nine or more diffraction peaks having 2θ angle values independently selected from the group consisting of 4.05±0.2, 8.00±0.2, 10.79±0.2, 11.23±0.2, 11.80±0.2, 12.02±0.2, 12.45±0.2, 12.95±0.2, 13.92±0.2, 15.55±0.2, 16.03±0.2, 16.83±0.2, 17.14±0.2, 17.76±0.2, 18.41±0.2, 19.38±0.2, 20.08±0.2, 20.50±0.2, 21.04±0.2, 21.66±0.2, 22.08±0.2, 22.52±0.2, 23.34±0.2, 23.72±0.2, 24.09±0.2, 24.82±0.2, 25.31±0.2, 25.66±0.2, 26.02±0.2, 27.21±0.2, 27.78±0.2, 28.21±0.2, 28.53±0.2, 29.01±0.2, 31.06±0.2, 31.45±0.2, 32.28±0.2, 33.02±0.2, 34.67±0.2, 35.30±0.2, 36.02±0.2, and 38.00±0.2; or
(c) oxalate Crystalline Form C, characterized by a powder X-ray diffraction pattern comprising three, four, five, six, seven, eight, nine or more diffraction peaks having 2θ angle values independently selected from the group consisting of 3.80±0.2, 4.01±0.2, 8.00±0.2, 10.42±0.2, 11.31±0.2, 11.55±0.2, 12.02±0.2, 12.94±0.2, 13.91±0.2, 14.13±0.2, 14.61±0.2, 16.04±0.2, 16.93±0.2, 17.76±0.2, 18.43±0.2, 18.91±0.2, 19.91±0.2, 20.29±0.2, 20.96±0.2, 21.53±0.2, 21.87±0.2, 22.34±0.2, 22.62±0.2, 23.16±0.2, 23.55±0.2, 23.76±0.2, 24.18±0.2, 24.86±0.2, 25.39±0.2, 25.65±0.2, 26.04±0.2, 26.46±0.2, 26.96±0.2, 27.53±0.2, 28.03±0.2, 28.47±0.2, 29.02±0.2, 29.47±0.2, 29.70±0.2, 30.16±0.2, 30.98±0.2, 31.36±0.2, 31.87±0.2, 32.53±0.2, 33.38±0.2, 34.41±0.2, 35.63±0.2, 35.91±0.2, 36.59±0.2, 38.15±0.2, and 38.66±0.2; or
(d) oxalate Crystalline Form D, characterized by a powder X-ray diffraction pattern comprising three, four, five, six, seven, eight, nine or more diffraction peaks having 2θ angle values independently selected from the group consisting of 4.06±0.2, 8.03±0.2, 10.51±0.2, 10.84±0.2, 11.60±0.2, 12.06±0.2, 12.85±0.2, 14.04±0.2, 14.67±0.2, 16.08±0.2, 16.98±0.2, 17.36±0.2, 17.80±0.2, 18.36±0.2, 18.68±0.2, 19.15±0.2, 19.85±0.2, 20.20±0.2, 20.46±0.2, 21.20±0.2, 21.66±0.2, 21.97±0.2, 22.38±0.2, 23.31±0.2, 23.80±0.2, 24.14±0.2, 24.31±0.2, 24.93±0.2, 25.52±0.2, 26.77±0.2, 27.28±0.2, 27.48±0.2, 28.33±0.2, 28.75±0.2, 29.59±0.2, 30.10±0.2, 30.81±0.2, 31.20±0.2, 32.16±0.2, 32.61±0.2, 33.36±0.2, 36.05±0.2, 38.64±0.2, and 39.25±0.2.

Embodiment 11. The crystalline form of any one of Embodiments 5-10, which is
(a) oxalate Crystalline Form A, characterized by a powder X-ray diffraction pattern comprising diffraction peaks having 2θ angle values of 11.68±0.2, 15.36±0.2, and 21.61±0.2;
preferably having 2θ angle values of 10.55±0.2, 11.68±0.2, 12.31±0.2, 15.36±0.2, 21.05±0.2, and 21.61±0.2;
more preferably having 2θ angle values of 10.55±0.2, 11.68±0.2, 12.31±0.2, 15.36±0.2, 15.58±0.2, 21.05±0.2, 21.61±0.2, 21.91±0.2, and 23.48±0.2;
even more preferably having 2θ angle values of 10.55±0.2, 11.68±0.2, 12.31±0.2, 15.36±0.2, 15.58±0.2, 18.39±0.2, 19.81±0.2, 21.05±0.2, 21.61±0.2, 21.91±0.2, 23.48±0.2, and 25.44±0.2;
even more preferably having 2θ angle values of 7.69±0.2, 10.55±0.2, 11.68±0.2, 11.21±0.2, 12.31±0.2, 15.36±0.2, 15.58±0.2, 18.39±0.2, 19.81±0.2, 21.05±0.2, 21.61±0.2, 21.91±0.2, 22.49±0.2, 23.48±0.2, and 25.44±0.2; or
(b) oxalate Crystalline Form B, characterized by a powder X-ray diffraction pattern comprising diffraction peaks having 2θ angle values of 11.80±0.2, 12.02±0.2, and 16.03±0.2;
preferably having 2θ angle values of 8.00±0.2, 11.80±0.2, 12.02±0.2, 12.45±0.2, 16.03±0.2, and 20.50±0.2;
more preferably having 2θ angle values of 8.00±0.2, 10.79±0.2, 11.80±0.2, 12.02±0.2, 12.45±0.2, 16.03±0.2, 20.50±0.2, 23.72±0.2, and 26.02±0.2;
even more preferably having 2θ angle values of 8.00±0.2, 10.79±0.2, 11.80±0.2, 12.02±0.2, 12.45±0.2, 16.03±0.2, 16.83±0.2, 20.08±0.2, 20.50±0.2, 23.72±0.2, 24.09±0.2, and 26.02±0.2;
even more preferably having 2θ angle values of 8.00±0.2, 10.79±0.2, 11.80±0.2, 12.02±0.2, 12.45±0.2, 13.92±0.2, 15.55±0.2, 16.03±0.2, 16.83±0.2, 20.08±0.2, 20.50±0.2, 23.72±0.2, 24.09±0.2, 26.02±0.2, and 28.21±0.2; or
(c) oxalate Crystalline Form C, characterized by a powder X-ray diffraction pattern comprising diffraction peaks having 2θ angle values of 10.42±0.2, 12.94±0.2, and 20.29±0.2;
preferably having 2θ angle values of 10.42±0.2, 12.94±0.2, 16.04±0.2, 18.91±0.2, 19.91±0.2, and 20.29±0.2;
more preferably having 2θ angle values of 10.42±0.2, 12.02±0.2, 12.94±0.2, 16.04±0.2, 16.93±0.2, 18.91±0.2, 19.91±0.2, 20.29±0.2, and 24.18±0.2;
even more preferably having 2θ angle values of 10.42±0.2, 12.02±0.2, 12.94±0.2, 16.04±0.2, 16.93±0.2, 18.91±0.2, 19.91±0.2, 20.29±0.2, 20.96±0.2, 21.87±0.2, 23.16±0.2, and 24.18±0.2;
even more preferably having 2θ angle values of 10.42±0.2, 12.02±0.2, 12.94±0.2, 16.04±0.2, 16.93±0.2, 18.43±0.2, 18.91±0.2, 19.91±0.2, 20.29±0.2, 20.96±0.2, 21.53±0.2, 21.87±0.2, 23.16±0.2, 24.18±0.2, and 28.47±0.2; or
(d) oxalate Crystalline Form D, characterized by a powder X-ray diffraction pattern comprising diffraction peaks having 2θ angle values of 10.51±0.2, 20.20±0.2, and 20.46±0.2; preferably having 2θ angle values of 10.51±0.2, 12.06±0.2, 12.85±0.2, and 16.08±0.2, 20.20±0.2, 20.46±0.2;
more preferably having 2θ angle values of 10.51±0.2, 12.06±0.2, 12.85±0.2, 16.08±0.2, 16.98±0.2, 20.20±0.2, 20.46±0.2, 21.66±0.2, and 23.80±0.2;
even more preferably having 2θ angle values of 10.51±0.2, 10.84±0.2, 12.06±0.2, 12.85±0.2, 16.08±0.2, 16.98±0.2, 19.15±0.2, 19.85±0.2, 20.20±0.2, 20.46±0.2, 21.66±0.2, and 23.80±0.2;
even more preferably having 2θ angle values of 10.51±0.2, 10.84±0.2, 12.06±0.2, 12.85±0.2, 16.08±0.2, 16.98±0.2, 18.36±0.2, 19.15±0.2, 19.85±0.2, 20.20±0.2, 20.46±0.2, 21.20±0.2, 21.66±0.2, 22.38±0.2, and 23.80±0.2.

Embodiment 12. The crystalline form of any one of Embodiments 5-11, substantially characterized by a powder X-ray diffraction pattern selected from the group consisting of FIG. 1A, FIG. 2A, FIG. 3A, and FIG. 4A.

Embodiment 13. A process for the preparation of the crystalline form of any one of Embodiments 5-12, comprising:
Procedure (1) dissolving the oxalate salt according to embodiment 4 in EtOH, and evaporating slowly the resultant solution to give the crystalline form; or
Procedure (2) dissolving the oxalate salt according to embodiment 4 in DCM, and evaporating slowly the resultant solution to give the crystalline form; or
Procedure (3) suspending the oxalate salt according to embodiment 4 in CPME, and stirring the resultant mixture to give the crystalline form; or
Procedure (4) suspending the oxalate salt according to embodiment 4 in toluene, and stirring the mixture to give the crystalline form.

Embodiment 14. The process for the preparation of the crystalline form of embodiment 13, wherein the time of Procedure (1) is 0-10 days, preferably is 0-7 days, more preferably is 2-4 days, even more preferably is 1, 2, 3, 4 or 5 days;
the time of Procedure (2) 0-10 days, preferably is 0-7 days, more preferably is 2-4 days, even more preferably is 1, 2, 3, 4 or 5 days;
the time of Procedure (3) 0-10 days, preferably is 0-7 days, more preferably is 2-4 days, even more preferably is 1, 2, 3, 4, 5, 6 or 7 days;
the time of Procedure (4) 0-10 days, preferably is 0-7 days, more preferably is 2-4 days, even more preferably is 1, 2, 3, 4, 5, 6 or 7 days.

Embodiment 15. The process for the preparation of the crystalline form of embodiment 13-14, wherein the oxalate salt/EtOH (mg/ml) in Procedure (1) is 1:1 to 100:1, preferably is 1:1 to 50:1, more preferably is 20:1 to 40:1, even more preferably is 30:1;
The oxalate salt/DCM (mg/ml) in Procedure (2) is 1:1 to 100:1, preferably is 1:1 to 50:1, more preferably is 20:1 to 40:1, even more preferably is 30:1;
The oxalate salt/CPME (mg/ml) in Procedure (3) is 1:1 to 100:1, preferably is 20:1 to 80:1, more preferably is 40:1 to 70:1, even more preferably is 60:1;
The oxalate salt/toluene (mg/ml) in Procedure (4) is 1:1 to 100:1, preferably is 20:1 to 80:1, more preferably is 40:1 to 70:1, even more preferably is 60:1.

Embodiment 16. The process for the preparation of the crystalline form of embodiment 13-15, wherein the temperature of Procedure (1) is 0 to 50° C., preferably is 10 to 40° C., more preferably is 20 to 30° C., even more preferably is 20 to 25° C. or room temperature;
the temperature of Procedure (2) is 0 to 50° C., preferably is 10 to 40° C., more preferably is 20 to 30° C., even more preferably is 20 to 25° C. or room temperature;
the temperature of Procedure (3) is 0 to 50° C., preferably is 10 to 40° C., more preferably is 20 to 30° C., even more preferably is 20 to 25° C. or room temperature;
the temperature of Procedure (4) is 10 to 110° C., preferably is 30 to 100° C., more preferably is 60 to 90° C., even more preferably is 80° C. or room temperature.

Embodiment 17. The process for the preparation of the crystalline form of embodiment 13-16, wherein the starting material is selected from amorphous, type A, B, C, D; preferably is amorphous.

Embodiment 18. The process for the preparation of the crystalline form of embodiment 13-17, wherein the process further comprises:
Procedure (5): dissolve the free base of compound 3 in solvent, then add oxalic acid to obtain the oxalate salt, wherein compound 3 is

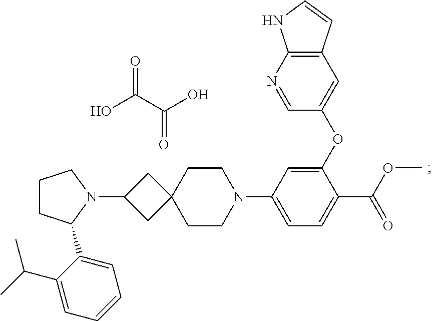

preferably, the solvent of Procedure (5) is methyl tert-butyl ether;
more preferably, oxalic acid is added by using the solution of methyl tert-butyl ether;
even more preferably, the temperature of Procedure (5) is room temperature or 20° C.-30° C.

Embodiment 19. The process of the crystalline form of any one of Embodiments 5-12 or the salt of any one of Embodiments 1-4, wherein the crystalline form or the salt is used as an intermediate of preparation of 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide.

### EXAMPLES

### Abbreviations

| | Abbreviations/ | |
|---|---|---|
| Category | Acronyms | Full Name/Description |
| Analytical | DSC | Differential scanning calorimetry |
| Techniques | DVS | Dynamic vapor sorption |
| | HPLC | High Performance Liquid |
| | | Chromatography |
| | NMR | Nuclear magnetic resonance |
| | TGA | Thermogravimetric analysis |
| | XRPD | X-ray powder diffraction |
| Solvent | MeOH | Methanol |
| | 2-MeTHF | 2-Methyltetrahydrofuran |
| | ACN | Acetonitrile |
| | CHCl₃ | Trichloromethane |
| | CPME | Cyclopentyl Methyl Ether |
| | DCM | Dichloromethane |
| | DMF | N,N-Dimethylformamide |
| | DMSO | Dimethylsulfoxide |
| | EtOAc, EA | Ethyl acetate |
| | EtOH | Ethanol |
| | IPA | Isopropyl alcohol |
| | IPAc | Isopropyl acetate |
| | MEK | Methyl ethyl ketone |
| | MIBK | Methyl isobutyl ketone |
| | MTBE | Methyl tert-butyl ether |
| | NMP | N-Methyl pyrrolido |
| | THF | Tetrahydrofuran |
| Agent | t-BuOK | Potassium tert-butoxide |
| | EDSA | 1,2-ethanedisulfonic acid |
| | D-DTTA | (+)-Di-1,4-toluoyl-D-tartaric acid |
| | NaOH | Sodium hydroxide |
| Others | aq. | Aqueous |
| | BE | Birefringence with Extinction |
| | Eq. | Equivalent |
| | IPC | In process control |
| | Vol or vol. | Volume |
| | w/w | Weight/weight |

### Instruments and Parameters

For XRPD analysis, a PANalytical Empyrean and X'Pert3 X-ray powder diffractometer were used to characterize the physical forms obtained in the present disclosure, without special instructions. The XRPD parameters used are listed as follows.

| Parameters | XRPD | |
|---|---|---|
| Model | Empyrean | X′ Pert³ |
| X-Ray wavelength | Cu, kα, | Cu, kα, |
| | Kα1 (Å): 1.540598, | Kα1 (Å): 1.540598, |
| | Kα2 (Å): 1.544426 | Kα2 (Å): 1.544426 |
| | Kα2/Kα1 intensity | Kα2/Kα1 intensity |
| | ratio: 0.50 | ratio: 0.50 |
| X-Ray tube setting | 45 kV, 40 mA | 45 kV, 40 mA |
| Divergence slit | Automatic | ⅛° |
| Scan mode | Continuous | Continuous |
| Scan range (°2Theta) | 3-40 | 3-40 |
| Scan step time (s) | 17.8 | 46.7 |
| Step size (°2TH) | 0.0167 | 0.0263 |
| Test Time (s) | 5 min 30 s | About 5 mins (5 min 04 s) |

For XRPD analysis, a Bruker D8 advanced X-Ray Powder diffractometer or equivalent was also used to characterize Form A and Form U. The XRPD parameters used are listed as follows.

| | |
|---|---|
| Tube | Cu, kα, Kα (Å): 1.540598, |
| Generator | Voltage: 40 kV; Current: 40 mA |
| Fixed incident beam optics | Primary Soller slit: 2.5 deg |
| | Secondary Soller slit: 2.5 deg |
| | Divergence Slit: 0.60 mm |
| | Slit: fixed |
| Detector | Detector Name: Lynxeye (1 D mode) |
| | PSD OPENING 2.1o |
| Scan mode | Continuous PSD fast |
| Scan range (°2Theta) | 4-40 deg |
| Scan type | Coupled Two Theta/Theta |
| Increment | 0.02 deg |
| Time/step | 0.12 s/step |

TGA and DSC were used to characterize the physical forms obtained in the present disclosure, without special instructions, wherein TGA data were collected using a TA Q500/Q5000 TGA from TA Instruments; and DSC was performed using a TA Q200/Q2000 DSC from TA Instruments. Detailed parameters used are listed as follows.

| Parameters | TGA | DSC | mDSC |
|---|---|---|---|
| Method | Ramp | Ramp | Modulated |
| Sample pan | Aluminum, open | Aluminum, | Aluminum, |
| | | crimped | crimped |
| Temperature | RT - desired | 25° C. - desired | 16° C. - desired |
| | temperature | temperature | temperature |
| Heating rate | 10° C./min | 10° C./min | 3° C./min |
| Purge gas | N2 | N2 | N2 |

For TGA and DGA analysis of Form A or U, some instruments were also used to conduct the testing, wherein TGA data were collected using a NETZSCH TG 209 F1 Instruments; and DSC was performed using a TA Q 20 or TA DSC 250 Instruments. Detailed parameters used are listed as follows.

| Parameters | TGA | DSC |
|---|---|---|
| Method | Ramp | Ramp |
| Sample pan | Aluminum, open | Aluminum, Sealed |
| Temperature | RT - desired temperature | RT - desired temperature |
| Heating rate | 10° C./min | 10° C./min |
| Purge gas | N2 | N2 |

DVS of the obtained forms in the present disclosure was measured via an SMS (Surface Measurement Systems) DVS Intrinsic, without special instructions (Method A). The relative humidity at 25° C. was calibrated against deliquescence point of LiCl, Mg(NO₃)₂ and KCl. Parameters for the DVS test are listed as follows.

| Parameters | DVS |
|---|---|
| Temperature | 25° C. |
| Sample size | 10~20 mg |
| Gas and flow rate | N2, 200 mL/min |
| dm/dt | 0.002%/min |
| Min. dm/dt stability | 10 min |
| duration | |
| Max. equilibrium time | 180 min |
| RH range | 70% RH-95% RH-0% RH-95% RH |
| RH step size | 10% (0% RH-90% RH, 90% RH-0% RH) |
| | 5% (90% RH-95% RH, 95% RH-90% RH) |

DVS of Form A and U was also measured via an SMS (Surface Measurement Systems) DVS Intrinsic (Method B). The relative humidity at 25° C. was calibrated against deliquescence point of LiCl, Mg(NO₃)₂, and KCl. Parameters for DVS test are listed as follows.

| Parameters | DVS |
|---|---|
| Temperature | 25° C. |
| Gas and flow rate | N2, 200 mL/min |
| dm/dt | <0.01 min |
| Min. dm/dt stability duration | 60 min |
| Max. equilibrium time | 180 min |
| RH range | Cycle: 40%-0%-95%-0%-40% RH |

The single crystal X-ray diffraction data were collected at 120 K using Rigaku XtaLAB Synergy R (CuK radiation, 1.54184 Å) diffractometer. The instrument parameters are listed as follows.

| Instrument | Rigaku XtaLAB Synergy R |
|---|---|
| X-Ray sources generator | MicroMax-007HF X-ray source |
| | (Cu/kα: 1.54184 Å) |
| Detector | HyPix 6000HE detector |
| Goniometer | Four-circle Kappa Goniometer |
| Low Temperature Devices | Cryostream-700 |
| | (Oxford Cryosystems) |
| Software package | CrysAlisPro (V1.171.40.14e) |

The following Examples are intended to further illustrate certain embodiments of the disclosure and are not intended to limit the scope of the disclosure.

Seven acids and seven organic solvents were used for the salt formation of Compound 2, see Table 1.

In one example, Compound 2 (1.0 g, 1.73 mmol) in MTBE (10 mL) solution and oxalic acid (0.16 g, 1.73 mmol) was added dropwise. The reaction mixture was stirred for about 6 hours and filtered to obtain a wet cake of methyl 4-(2-{(2S)-2-[2-(propan-2-yl)phenyl]pyrrolidin-1-yl}-7-azaspiro[3.5]nonan-7-yl)-2-[(1H-pyrrolo[2,3-b]pyridin-5-yl)oxy]benzoate oxalate salt (Compound 3). The solid has low hydroscopic property, also the solid has poor static electricity capacity so it's not easy to adsorb to objects.

Other salts of Compound 2 was produced by a procedure similar to that disclosed for Compound 3 by using other acids. The other acids were used as the following amounts, D-(−)-tartaric (0.26 g, 1.73 mmol), p-toluenesulfonic acid (0.3 g, 1.73 mmol), D-DTTA (0.67 g, 1.73 mmol), Citric acid (0.33 g, 1.73 mmol), Malic acid (0.23 g, 1.73 mmol), Maleic acid (0.20 g, 1.73 mmol); and the other organic solvents were used with 10 mL volume.

The corresponding physical forms of Compound 2 salt was recited in Table 1. The result showed that, when the acid used in salt formation was Oxalic acid or D-DTTA, the obtained salt was solid form in certain organic solvents. Considering simplify the process D-DTTA/MTBE and Oxalic acid/MTBE were preferred as the salt formation systems, while D-DTTA (M.W. 386.35 g/mol) may cause more waste to the reaction compared with Oxalic acid (M.W. 90.03 g/mol).

TABLE 1

| | Acid | | | | | | |
|---|---|---|---|---|---|---|---|
| | | | p-toluenesulfonic | | | | |
| Solvent | D-(—)-tartaric | Oxalic acid | acid | D-DTTA | Citric acid | Malic acid | Maleic acid |
| 2-MeTHF | oil | oil | Clean solution | Clean solution | oil | Clean solution | Clean solution |
| MTBE | moisture | Fine solid | moisture absorption | Fine solid | moisture | moisture | moisture |
| | absorption | | | | absorption | absorption | absorption |
| IPAc | moisture | moisture | Clean solution | oil | moisture | oil | oil |
| | absorption | absorption | | | absorption | | |
| Acetone | Clean solution | Clean solution | Clean solution | Clean solution | Clean | Clean solution | Clean solution |
| | | | | | solution | | |
| MeOH | Clean solution | Clean solution | Clean solution | Clean solution | Clean | Clean solution | Clean solution |
| | | | | | solution | | |
| EA | oil | oil | Clean solution | Clean solution | oil | oil | Clean solution |
| DCM | Clean solution | Clean solution | Clean solution | Clean solution | Clean | Clean solution | Clean solution |
| | | | | | solution | | |

### Synthesis of Compound-3

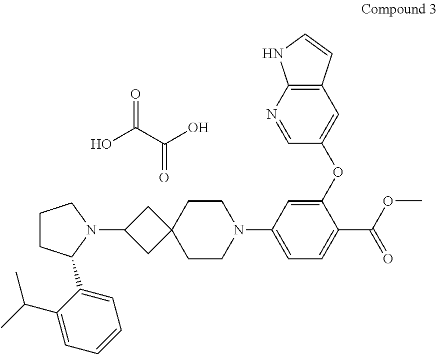

Free base of 3 (21 g) was dissolved in methyl tert-butyl ether (110 g). The temperature was adjusted to 20° C.-30° C., then the solution of oxalic acid (3.33 g) in methyl tert-butyl ether (110 g) was added within 2 h. The solution was stirred at 20° C.-30° C. for 16 h. The resulting solid was filtered, washed with methyl tert-butyl ether (20 g). The filtered solid was dried at a temperature below 45° C. for 20-28 h as amorphous.

### Example 1A: Preparation of Compound 3 Form A (Form A)

Crystalline Form A of Compound 3 sample was obtained via slow evaporation of a solution of Compound 3 in amorphous form in EtOH at RT.

Procedure: 30 mg of an amorphous solid of Compound 3 was weighed into a 3-mL glass vial, and 1 mL of EtOH was added into the vial to get a clear solution. The solution was evaporated at RT for 3 days to induce precipitation to obtain Crystalline Form A.

The X-ray powder diffraction (XRPD) pattern (conducted on Bruker D8 advanced X-Ray Powder diffractometer) was used to characterize the obtained Form A, which showed that Form A was in a crystalline form, see FIG. 1A. The characteristic peaks and percent peak intensities obtained from the XRPD analysis are listed in Table 1A.

TABLE 1A
XRPD pattern of Compound 3 Form A

| Pos. [°2θ] | d-spacing [Å] | Rel. Int. [%] |
|---|---|---|
| 3.89 | 22.68 | 4.5 |
| 7.69 | 11.49 | 30.7 |
| 10.55 | 8.38 | 55.7 |
| 10.84 | 8.16 | 13.5 |
| 11.21 | 7.89 | 24.4 |
| 11.68 | 7.57 | 95.9 |
| 12.31 | 7.18 | 70.2 |
| 13.02 | 6.79 | 19.7 |
| 13.83 | 6.40 | 9.3 |
| 14.69 | 6.03 | 9.2 |
| 15.36 | 5.77 | 100 |
| 15.58 | 5.68 | 50.9 |
| 16.60 | 5.34 | 17.1 |
| 17.58 | 5.04 | 17.1 |
| 18.39 | 4.82 | 41.3 |
| 18.98 | 4.67 | 19.2 |
| 19.32 | 4.59 | 9.7 |
| 19.81 | 4.48 | 40.5 |
| 20.37 | 4.36 | 9.7 |
| 21.05 | 4.22 | 65.6 |
| 21.61 | 4.11 | 90.4 |
| 21.91 | 4.05 | 46.7 |
| 22.49 | 3.95 | 24.8 |
| 23.09 | 3.85 | 18.3 |
| 23.48 | 3.79 | 53.4 |
| 24.15 | 3.68 | 2.9 |
| 24.76 | 3.59 | 24.1 |
| 25.44 | 3.50 | 45.9 |
| 25.66 | 3.47 | 22.2 |
| 26.18 | 3.40 | 1.3 |
| 26.67 | 3.34 | 2.5 |
| 27.00 | 3.30 | 6.4 |
| 28.11 | 3.17 | 5 |
| 28.71 | 3.11 | 11.3 |
| 29.09 | 3.07 | 7.6 |
| 29.55 | 3.02 | 4.2 |
| 29.91 | 2.99 | 0.6 |
| 31.52 | 2.84 | 3.1 |
| 32.18 | 2.78 | 8.5 |
| 32.80 | 2.73 | 9.2 |
| 34.49 | 2.60 | 2.4 |
| 34.97 | 2.56 | 2.1 |
| 36.66 | 2.45 | 1.3 |
| 38.52 | 2.34 | 2.7 |
| 39.32 | 2.29 | 2 |
| 39.71 | 2.27 | 1.9 |

TGA result indicated 2.7% of weight loss from 100 to 150° C. and 14% of weight loss from 160 to 250° C. DSC result showed three endothermic peaks at 51° C., 114° C. and 192° C. (onset temperature), respectively (FIG. 1B). In the ¹H NMR spectrum, about 0.27% of EtOH was observed (FIG. 1C).

### Example 2A: Preparation of Compound 3 Form B (Form B)

Crystalline Form B of Compound 3 sample was obtained via slow evaporation of a solution of Compound 3 in an amorphous form in DCM at RT.

Procedure: 30 mg of amorphous solid of Compound 3 was weighed into a 3-mL glass vial, and 1 mL of DCM was added into the vial to get a clear solution. The solution was evaporated at RT to induce precipitation to obtain Crystalline Form B.

The XRPD pattern was used to characterize the obtained Form B which showed that Form B was in a crystalline, see FIG. 2A. The characteristic peaks and percent peak intensities obtained from the XRPD analysis are listed in Table 2A.

TABLE 2A
XRPD pattern of Compound 3 Form B

| Pos. [°2θ] | d-spacing [Å] | Rel. Int. [%] |
|---|---|---|
| 4.05 | 21.81 | 6.9 |
| 8.00 | 11.04 | 24.8 |
| 10.79 | 8.20 | 20.5 |
| 11.23 | 7.87 | 2.1 |
| 11.80 | 7.49 | 64.7 |
| 12.02 | 7.36 | 66.2 |
| 12.45 | 7.10 | 23.3 |
| 12.95 | 6.83 | 5.7 |
| 13.92 | 6.35 | 14.3 |
| 15.55 | 5.69 | 13.4 |
| 16.03 | 5.53 | 100 |
| 16.83 | 5.27 | 15.1 |
| 17.14 | 5.17 | 6 |
| 17.76 | 4.99 | 1.9 |
| 18.41 | 4.81 | 11.8 |
| 19.38 | 4.58 | 6.7 |
| 20.08 | 4.42 | 16.7 |
| 20.50 | 4.33 | 29.9 |
| 21.04 | 4.22 | 6.4 |
| 21.66 | 4.10 | 1.1 |
| 22.08 | 4.02 | 9.9 |
| 22.52 | 3.94 | 10.6 |
| 23.34 | 3.81 | 4 |
| 23.72 | 3.75 | 20.6 |
| 24.09 | 3.69 | 18.4 |
| 24.82 | 3.58 | 7.5 |
| 25.31 | 3.52 | 6.7 |
| 25.66 | 3.47 | 13.1 |
| 26.02 | 3.42 | 18.9 |
| 27.21 | 3.27 | 2.1 |
| 27.78 | 3.21 | 5.2 |
| 28.21 | 3.16 | 15 |
| 28.53 | 3.13 | 10.1 |
| 29.01 | 3.08 | 2.2 |
| 31.06 | 2.88 | 6.9 |
| 31.45 | 2.84 | 4.3 |
| 32.28 | 2.77 | 1.7 |
| 33.02 | 2.71 | 2.5 |
| 34.67 | 2.59 | 1.6 |
| 35.30 | 2.54 | 1.1 |
| 36.02 | 2.49 | 0.8 |
| 38.00 | 2.37 | 1.9 |

TGA result indicated 0.9% of weight loss from 100 to 150° C. and 8.6% of weight loss from 170 to 250° C. DSC result showed three endothermnic peaks at 53° C., 124° C. and 192° C. (onset temperature), respectively (FIG. 2B). In the ¹H NMR spectrum, about 3% of DCM was observed (FIG. 2C).

### Example 3A: Preparation of Compound 3 Form C (Form C)

Crystalline Form C of Compound 3 sample was obtained via slurrying Compound 3 in an amorphous form in CPME at RT.

Procedure: 30 mg of amorphous solid of Compound 3 was weighed into a 3-mL glass vial, and 0.5 mL of CPME was added into the vial to get a suspension. The mixture was stirred at RT magnetically with a speed of 800 RPM for 4 days to obtain Crystalline Form C.

The XRPD pattern was used to characterize the obtained Form C which showed that Form C was in a crystalline, see FIG. 3A. The characteristic peaks and percent peak intensities obtained from the XRPD analysis are listed in Table 3A.

TABLE 3A
XRPD pattern of Compound 3 Form C

| Pos. [°2θ] | d-spacing [Å] | Rel. Int. [%] |
|---|---|---|
| 3.80 | 23.22 | 4.9 |
| 4.01 | 22.01 | 12.4 |
| 8.00 | 11.05 | 8.6 |
| 10.42 | 8.48 | 100 |
| 11.31 | 7.82 | 1.7 |
| 11.55 | 7.66 | 6.4 |
| 12.02 | 7.36 | 42.9 |
| 12.94 | 6.84 | 59.8 |
| 13.91 | 6.36 | 3.8 |
| 14.13 | 6.26 | 5.1 |
| 14.61 | 6.06 | 1.4 |
| 16.04 | 5.52 | 46 |
| 16.93 | 5.23 | 33.3 |
| 17.76 | 4.99 | 14.3 |
| 18.43 | 4.81 | 15.9 |
| 18.91 | 4.69 | 47.2 |
| 19.91 | 4.46 | 45.1 |
| 20.29 | 4.37 | 59.4 |
| 20.96 | 4.23 | 16.5 |
| 21.53 | 4.12 | 15.8 |
| 21.87 | 4.06 | 22.8 |
| 22.34 | 3.98 | 13.7 |
| 22.62 | 3.93 | 7.7 |
| 23.16 | 3.84 | 17.3 |
| 23.55 | 3.77 | 10 |
| 23.76 | 3.74 | 14.7 |
| 24.18 | 3.68 | 32.5 |
| 24.86 | 3.58 | 3.2 |
| 25.39 | 3.51 | 14.6 |
| 25.65 | 3.47 | 8.1 |
| 26.04 | 3.42 | 0.5 |
| 26.46 | 3.37 | 4.6 |
| 26.96 | 3.30 | 4.7 |
| 27.53 | 3.24 | 1.1 |
| 28.03 | 3.18 | 11.1 |
| 28.47 | 3.13 | 15.1 |
| 29.02 | 3.07 | 1.5 |
| 29.47 | 3.03 | 1.6 |
| 29.70 | 3.01 | 4.5 |
| 30.16 | 2.96 | 2.3 |
| 30.98 | 2.88 | 3.3 |
| 31.36 | 2.85 | 9.7 |
| 31.87 | 2.81 | 1.7 |
| 32.53 | 2.75 | 1.8 |
| 33.38 | 2.68 | 7.6 |
| 34.41 | 2.60 | 2.9 |
| 35.63 | 2.52 | 4.1 |
| 35.91 | 2.50 | 3 |
| 36.59 | 2.45 | 3.1 |
| 38.15 | 2.36 | 2.1 |
| 38.66 | 2.33 | 1.7 |

TGA result indicated 4.1% of weight loss from 95 to 150° C. and 14.8% of weight loss from 150 to 250° C. DSC result showed two endothermic peaks at 131° C. and 194° C. (onset temperature), respectively (FIG. 3B). In the ¹H NMR spectrum, about 5.9% of CPME was observed (FIG. 3C).

### Example 4A: Preparation of Compound 3 Form D (Form D)

Crystalline Form D of Compound 3 sample was obtained via slurrying Compound 3 in an amorphous form in toluene at 80° C.

Procedure: 30 mg of amorphous solid of Compound 3 was weighed into a 3-mL glass vial, and 0.5 mL of toluene was added into the vial to get a suspension. The mixture was stirred at 80° C. magnetically with a speed of 800 RPM for 4 days to obtain Crystalline Form D.

The XRPD pattern was used to characterize the obtained Form D which showed that Form D was in a crystalline, see FIG. 4A. The characteristic peaks and percent peak intensities obtained from the XRPD analysis are listed in Table 4A.

TABLE 4A
XRPD pattern of Compound 3 Form D

| Pos. [°2θ] | d-spacing [Å] | Rel. Int. [%] |
|---|---|---|
| 4.06 | 21.74 | 5.7 |
| 8.03 | 11.00 | 13.4 |
| 10.51 | 8.41 | 100 |
| 10.84 | 8.16 | 25 |
| 11.60 | 7.62 | 2.1 |
| 12.06 | 7.33 | 47.8 |
| 12.85 | 6.88 | 52.4 |
| 14.04 | 6.30 | 8.2 |
| 14.67 | 6.03 | 2 |
| 16.08 | 5.51 | 43.8 |
| 16.98 | 5.22 | 32.8 |
| 17.36 | 5.10 | 2.6 |
| 17.80 | 4.98 | 19.9 |
| 18.36 | 4.83 | 25 |
| 18.68 | 4.75 | 6.7 |
| 19.15 | 4.63 | 30.3 |
| 19.85 | 4.47 | 31.8 |
| 20.20 | 4.39 | 68.7 |
| 20.46 | 4.34 | 55.5 |
| 21.20 | 4.19 | 20.4 |
| 21.66 | 4.10 | 39.6 |
| 21.97 | 4.04 | 13.2 |
| 22.38 | 3.97 | 21 |
| 23.31 | 3.81 | 16 |
| 23.80 | 3.74 | 38.3 |
| 24.14 | 3.68 | 15.9 |
| 24.31 | 3.66 | 11.4 |
| 24.93 | 3.57 | 3.6 |
| 25.52 | 3.49 | 14.4 |
| 26.77 | 3.33 | 6.2 |
| 27.28 | 3.27 | 5.1 |
| 27.48 | 3.24 | 5 |
| 28.33 | 3.15 | 17.2 |
| 28.75 | 3.10 | 7.3 |
| 29.59 | 3.02 | 3.8 |
| 30.10 | 2.97 | 5.5 |
| 30.81 | 2.90 | 2.5 |
| 31.20 | 2.86 | 11.2 |
| 32.16 | 2.78 | 1.2 |
| 32.61 | 2.74 | 1.6 |
| 33.36 | 2.68 | 9.1 |
| 36.05 | 2.49 | 2 |
| 38.64 | 2.33 | 2 |
| 39.25 | 2.29 | 0.8 |

TGA result indicated 3.1% of weight loss from 120 to 170° C. and 13.5% of weight loss from 170 to 250° C. DSC result showed two endothermic peaks at 132° C. and 194° C. (onset temperature), respectively (FIG. 4B). In the ¹H NMR spectrum, about 5.2% of toluene was observed (FIG. 4C).

### Example 5: Sonrotoclax

### Step 1: (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid

Methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate was synthesized per the methods in WO2019210828 (e.g., Example F43), which is incorporated by reference in its entirety.

To a solution of methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate (105 g, 181.7 mmol) in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5˜6 with 3 N HCl acid at room temperature with stirring. The precipitate was filtered and dried in vacuum to give the product as a white solid (102.4 g, yield: 99%). ¹H NMR (400 MHz, DMSO-d₆) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1H), 2.9-2.15 (m, 1H), 1.74-1.64 (m, 4H), 1.52-1.37 (m, 6H), 1.28-1.06 (in, 6H). MS (ESI, m/e) [M+1]⁺ 564.9.

### Step 2: Sonrotoclax

A mixture of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol) in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid was consumed completely, the reaction mixture was heated to −35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to room temperature, the precipitate was filtered and then the wet cake was washed twice with EA (180 mL). After drying in vacuum at 80-90° C., sonrotoclax was obtained (48 g, yield: 69.5%). ¹H NMR (DMSO-d₆) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]⁺ 889.9.

What is claimed is:

1. A crystalline form of Compound 3, 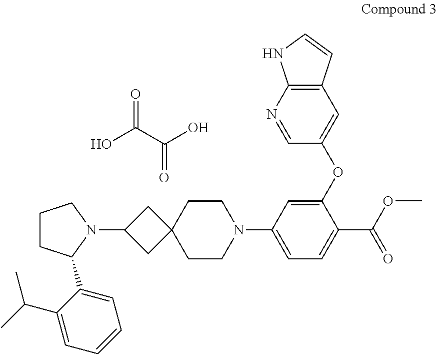
substantially characterized by a powder X-ray diffraction pattern substantially in accordance with that shown in a figure selected from the group consisting of FIG. 1A, FIG. 2A, FIG. 3A, and FIG. 4A.

2. The crystalline form of claim 1, wherein the powder X-ray diffraction pattern is substantially in accordance with that shown in FIG. 1A.

3. The crystalline form of claim 1, wherein the powder X-ray diffraction pattern is substantially in accordance with that shown in FIG. 2A.

4. The crystalline form of claim 1, wherein the powder X-ray diffraction pattern is substantially in accordance with that shown in FIG. 3A.

5. The crystalline form of claim 1, wherein the powder X-ray diffraction pattern is substantially in accordance with that shown in FIG. 4A.

6. A method for preparing sonrotoclax, 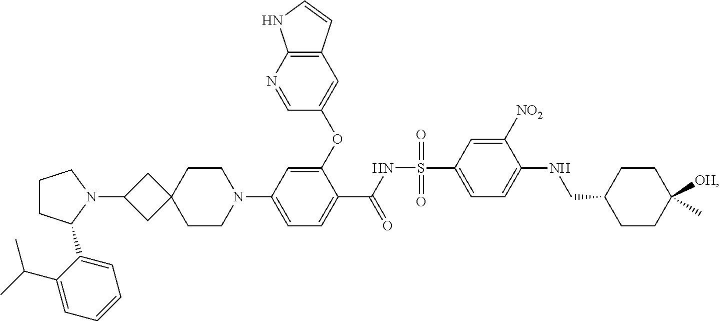 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof, comprising reacting a salt of formula (I) 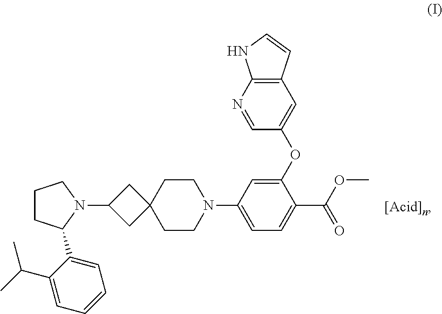 [SMILES: COC(=O)c1ccc(N2CCC3(CC2)CC(N2CCC[C@H]2c2ccccc2C(C)C)C3)cc1Oc1cnc2[nH]ccc2c1]
with 4-((((1r,4r)-4-hydroxy-4-methylcyclohexyl)methyl)amino)-3-nitrobenzenesulfonamide, wherein [Acid] is selected from hydrochloric acid, sulfuric acid, phosphoric acid, hydrobromic acid, nitric acid, fumaric acid, tartaric acid, lauric acid, stearic acid, gentian acid, niacin, aspartic acid, succinic acid, adipic acid, malic acid, citric acid, glycolic acid, gluconic acid, lactic acid, acetic acid, benzenesulfonic acid, methanesulfonic acid, benzoic acid, naphthalenesulfonic acid, p-toluenesulfonic acid, (+)-di-1,4-toluoyl-D-tartaric acid (D-DTTA), maleic acid or oxalic acid; and
n is about 0.5 to about 3.

7. The method of claim 6, comprising reacting the salt of formula (I) with 4-((((1r,4r)-4-hydroxy-4-methylcyclohexyl)methyl)amino)-3-nitrobenzenesulfonamide in the presence of 1-ethyl-3-(3-dimethylaminopropyl)carbodiimide (EDCI) and 4-dimethylaminopyridine (DMAP).

8. The method of claim 6, wherein the salt of formula (I) is the crystalline form of claim 1.

9. A method for preparing sonrotoclax, 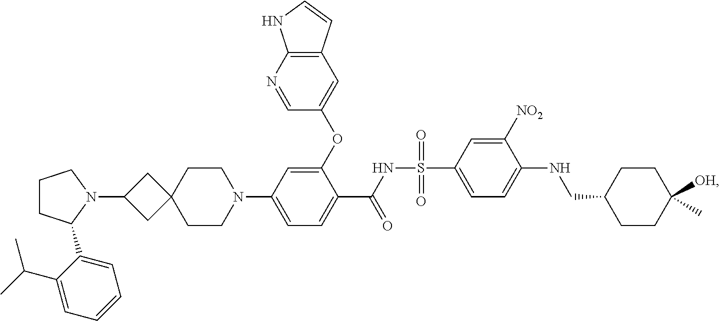 [SMILES: CC(C)c1ccccc1[C@@H]1CCCN1C1CC2(CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)C1]
or a salt thereof, comprising reacting the crystalline form according to claim 1, with an acid or a base, to provide (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, 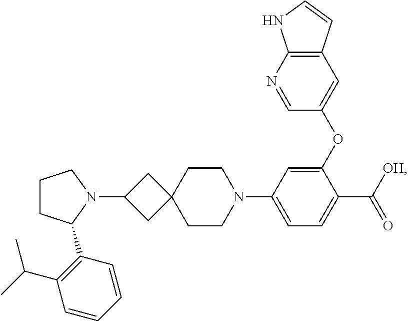 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof.

10. The method of claim 9, wherein the acid is HCl, or the base is NaOH.

11. The method of claim 9, further comprising reacting (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, 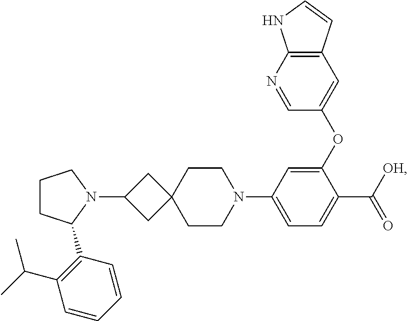 [SMILES: CC(C)c1ccccc1[C@@H]1CCCN1C1CC2(CCN(c3ccc(C(=O)O)c(Oc4cnc5[nH]ccc5c4)c3)CC2)C1]
or a salt thereof, with 4-((((1r,4r)-4-hydroxy-4-methylcyclohexyl)methyl)amino)-3-nitrobenzenesulfonamide.

12. The method of claim 11, comprising reacting (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid, or a salt thereof, with 4-((((1r,4r)-4-hydroxy-4-methylcyclohexyl)methyl)amino)-3-nitrobenzenesulfonamide in the presence of EDCI and DMAP.

13. A method for preparing a pharmaceutical composition comprising sonrotoclax, comprising mixing sonrotoclax with a pharmaceutically acceptable excipient, wherein sonrotoclax is prepared according to the method of any one of claims 6-12.

14. A pharmaceutical composition comprising sonrotoclax or a pharmaceutically acceptable salt thereof and a pharmaceutically acceptable excipient, wherein sonrotoclax is prepared according to the method of any one of claims 6-11.
